(19) United States
(12) Patent Application Publication
(10) Pub. No.: US 2016/0287624 A1
(43) Pub. Date: Oct. 6, 2016

(54) Water-Soluble Vitamins to Increase Oral Absorption and Bioavailability

(71) Applicants: NESTEC S.A., Vevey (CH); BOARD OF SUPERVISORS OF LOUISIANA STATE UNIVERSITY AND AGRICULTURAL AND MECHANICAL COLLEGE, Baton Rouge, LA (US)

(72) Inventors: Zhijun Liu, Baton Rouge, LA (US); Juan A. Ruiz, Covington, LA (US); Richard Copp, Covington, LA (US); Harold Koch, JR., Covington, LA (US)

(21) Appl. No.: 15/037,962

(22) PCT Filed: Nov. 20, 2014
(86) PCT No.: PCT/US2014/066718

§ 371 Date: May 19, 2016

Related U.S. Application Data
(60) Provisional application No. 61/907,275, filed on Nov. 21, 2013.

(51) Int. Cl. A61K 31/714; A61K 9/16; A61K 9/00; A61K 31/704

(52) U.S. Cl. CPC A61K 31/714; A23L 1/302; A23L 1/2366; A61K 31/704; A61K 9/16; A61K 9/0053; A23V 2002/00

(57) ABSTRACT

The present invention provides formulations for enhancing bioavailability of water-soluble compounds including water-soluble vitamins. The formulations include a water-soluble vitamin and a glycoside selected from a diterpene glycoside and a triterpene glycoside. Methods for increasing the bioavailability of a water-soluble vitamin are also described

### CROSS-REFERENCES TO RELATED APPLICATIONS

The present application claims priority to U.S. Provisional Patent Application No. 61/907,275, filed Nov. 21, 2013, the teachings of which are hereby incorporated by reference in their entirety for all purposes.

### BACKGROUND OF THE INVENTION

Vitamin B₁₂ is a water-soluble cofactor for two human enzymes methionine synthase and L-methylmalonyl-coenzyme A mutase (MCM). Methionine synthase converts homocysteine to methionine, which is essential for nucleic acid synthesis, and MCM activity is required for proper function of several metabolic pathways. Vitamin B₁₂ deficiency can cause megalobastic anemia, bone marrow failure, demyelinating nervous system disease, autoimmune gastritis (pernicious anemia), glossitis, infertility, thrombosis, and other conditions.

Vitamin B₁₂ deficiency can result from dietary deficiency or from a lack of intrinsic factor, a glycoprotein produced by the stomach that binds to the vitamin and enables its absorption by the intestine. To overcome the insufficient absorption in the latter case, vitamin B₁₂ must be administered to a deficient patient by parenteral/intramuseular injection or by high-dose oral treatment. Intramuscular injection can discourage proper compliance in vitamin B₁₂-deficient patients, and oral treatment can be ineffective in the most severe cases of vitamin B₁₂ deficiency. Therefore, new methods for efficient vitamin B₁₂ administration that encourage patient compliance are needed. The present invention addresses this need, providing formulations and methods that increase the bioavailability of vitamin B₁₂ and other water-soluble compounds.

### BRIEF SUMMARY OF THE INVENTION

The present invention relates to compositions and methods for improving absorption and bioavailability. More particularly, the present invention relates to compositions of a biologically active substance such as a vitamin together with a glycoside to increase the extent to which the substance is absorbed into the bloodstream of a subject after administration.

As such, in one embodiment, the present invention provides a formulation for enhancing bioavailability of vitamin B₁₂, the formulation comprising, consisting essentially of, or consisting of:
vitamin B₁₂; and
a glycoside selected from the group of a diterpene glycoside or a triterpene glycoside.

In certain aspects, the vitamin B₁₂ is a cobalamin selected from the group of cyanocobalamin, hydroxocobalamin, methylcobalamin, 5-deoxyadenosylcobalamin. Preferably, the vitamin B₁₂ is methylcobalamin.

In certain aspects, the formulation is substantially free of intrinsic factor.

In certain other aspects, the glycoside is a diterpene glycoside.

In another embodiment, the present invention provides a method for making a formulation of vitamin B₁₂, the method comprising, consisting essentially of, or consisting of:
admixing vitamin B₁₂ and a glycoside selected from the group of a diterpene glycoside or triterpene glycoside in a solvent to form a vitamin B₁₂ admixture;
heating the vitamin B₁₂ admixture to form a clear vitamin B₁₂ admixture; and
removing the solvent from the clear vitamin B₁₂ admixture to form the formulation of vitamin B₁₂.

In yet another embodiment, the present invention provides a method for increasing the bioavailability of vitamin B₁₂, the method comprising, consisting essentially of, or consisting of:
administering a formulation comprising vitamin B₁₂; and a glycoside selected from the group consisting of a diterpene glycoside and a triterpene glycoside, thereby increasing the bioavailability of vitamin B₁₂.

These and other aspects, objects and advantages will become more apparent when read with the detailed description of the invention and the drawings which follow.

### BRIEF DESCRIPTION OF THE DRAWINGS

FIG. 1A shows the structure of rubusoside. FIG. 1B shows the structure of stevioside. FIG. 1C shows the structure of rebaudioside A.

FIG. 2A and FIG. 2B show the structures of several diterpene glycosides isolated from Rubus or Stevia plants.

FIG. 3 shows the concentration of API (vitamin B₁₂) in rat plasma over time after oral gavage with baseline normalized for all treatments. Rats (n=6 per group) were orally administered at a dose equivalent to 2 mg/70 kg person in a free form (unformulated control F4) or a formulation of Formula 1 (API:Stevia at 1:25 w/w), Formula 2 (API:Stevia at 1:50 w/w), or Formula 3 (API:Stevia at 1:125 w/w). Vertical bars across each data point represent one standard error.

### DETAILED DESCRIPTION OF THE INVENTION

### I. DEFINITIONS

As used herein, the term “formulation” includes compositions containing a biologically active substance such as a vitamin together with a glycoside. The formulation can include one or more pharmaceutically acceptable excipients.

As used herein, the term “bioavailability” includes the extent to which a substance is absorbed into the bloodstream of a subject after administration of a pharmaceutical formulation, and the amount of the substance that reaches the general circulation of the subject.

As used herein, the terms “enhancing bioavailability” and “increasing bioavailability” include administering a substance, such as a vitamin, so as to raise the bioavailability of the substance above the level at which it would be normally available. Administering the substance can include formulating the substance so as to increase the bioavailability. The substance can be formulated to increase the bioavailability by any suitable amount. In general, the methods of the present invention lead to bioavailability increases of at least about 10%, as compared to administration via control methods. Bioavailability levels can be determined by any suitable method, including analysis of the drug in a blood, plasma, serum, or urine sample taken from a subject after administration. Bioavailability can be assessed, for example, by plotting the concentration of a substance in the circulation of a subject over time after administration. Bioavailability can be considered in terms of the maximum (peak) concentration of the substance in the blood after administration, as well as in terms of the time required for the concentration of the substance to reach the peak concentration. The “area under the curve” (AUC) of the concentration-vs.-time plot can be calculated and used to determine the total amount of the substance that is absorbed into the blood stream after administration of a single dose.

As used herein, the term “water-soluble vitamin” includes vitamin C or a B vitamin.

As used herein, the term “B vitamin” includes thiamine (vitamin B₁), riboflavin, niacin/nicotinic acid/nicotinamide (vitamin B₃), folic acid, folinic acid, L-methylfolate, L-5 methylfolate (vitamin B₉), pyridoxine/pyridoxal/pyridoxamine (vitamin B₆), biotin (vitamin B₇), pantothenic acid (vitamin B), and vitamin B₁₂.

As used herein, the term “vitamin B₁₂” includes a cobalamin compound according to Formula I:

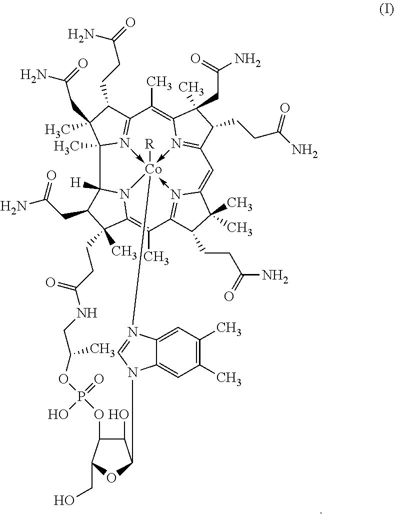

For cobalamin compounds according to Formula I, the oxidation state of the cobalt atom is +1, +2, or +3 depending on the identity of the ligand R. R can be absent in the compounds of Formula I. When R is absent, the compound is referred to as cobalamin. Alternatively, R is selected from methyl, cyano, and 5′-deoxyadenosyl. When R is methyl, the compound is referred to as methylcobalamin. When R is cyano, the compound is referred to as cyanocobalamin. When R is 5′-deoxyadenosyl, the compound is referred to as 5′-deoxyadenosylcobalamin. Vitamin B₁₂ can also refer to a mixture of the cobalamin compounds described herein. Vitamin B₁₂ can also refer to pharmaceutically acceptable salts of the cobalamin compounds described herein.

As used herein, the term “vitamin B₆” includes pyridoxine, pyridoxal, pyridoxamine, and pharmaceutically acceptable salts thereof.

As used herein, the term “vitamin C” includes L-ascorbic acid, also known as (R)-3,4-dihydroxy-5-((S)-1,2-dihydroxyethyl)furan-2(5H)-one, and pharmaceutically acceptable salts thereof.

As used herein, the term “terpene” refers to an organic compound having one or more isoprene-derived subunits. Terpenes are generally synthesized chemically or biochemically from isoprene (2-methyl-1,3-butadiene having the formula CH₂C(CH₃)CHCH₂, i.e., C₅H₈) and isoprene derivatives. A “monoterpene” is generally understood to contain two isoprene subunits and has a base molecular formula of C₁₀H₁₆. “Diterpenes” and “triterpenes” typically contain four and six isoprene subunits, respectively. Terpenes, including diterpenes and triterpenes, can contain isoprene subunits arranged in a linear or cyclic configuration. The linear or cyclic backbones can be substituted with one or more moieties including, but not limited to, hydroxy, oxo, and carboxy groups.

As used herein, the term “glycoside” includes a compound having one or more sugar moieties and a non-sugar moiety. The sugar moieties generally contain from 1 to 6 monosaccharide subunits having from 5 to 6 carbon atoms. Examples of typical monosaccharide subunits include, but are not limited to, glucose, allose, altrose, mannose, gulose, idose, galactose, talose, psicose, fructose, sorbose, tagatose, arabinose, lyxose, ribose, xylose, ribulose, and xylulose. The monosaccharide subunits can also be deoxy sugars, amino sugars, or sulfosugars. The monosaccharide subunits can be linked to each other in a number of configurations. For example, linkages can occur between the 1-carbon (the anomeric carbon) and the 4-carbon of adjacent monosaccharide subunits (i.e., a 1-4 linkage), the 1-carbon and the 3-carbon of adjacent monosaccharide subunits (i.e., a 1-3 linkage), the 1-carbon and the 6-carbon of adjacent monosaccharide subunits (i.e., a 1-6 linkage), or the 1-carbon and the 2-carbon of adjacent monosaccharide subunits (i.e., a 1-2 linkage). A monosaccharide subunit can be linked within a sugar moiety such that the anomeric carbon is in the α- or β-configuration. The sugar moieties can also include linkages between carbon atoms other than the 1-, 2-, 3-, 4-, and 6-carbons. The non-sugar moiety in a glycoside (“the aglycone”) is typically connected to a sugar moiety via an ether linkage.

As used herein, the term “diterpene glycoside” includes glycosides as defined above, wherein the non-sugar moiety is a diterpene. Examples of diterpene glycosides include, but are not limited to, rebaudiosides, suaviosides, goshonosides, paniculosides, stevioside, and rubososide. A stevioside is preferred.

As used herein, the term “triterpene glycoside” includes glycosides as defined above, wherein the non-sugar moiety is a triterpene. Examples of triterpene glycosides include, but are not limited to, abrusosides, cimiracemosides, lansiosides, leucospilotasides, frondoside A, eximisoside A, and quadranguloside.

As used herein, the term “stevioside” (CAS 57817-89-7) refers to (4α)-13-[(2-O-β-D-glucopyranosyl-β-D-glucopyranosyl)oxy]kaur-16-en-18-oic acid β-D-glucopyranosyl ester having the structure:

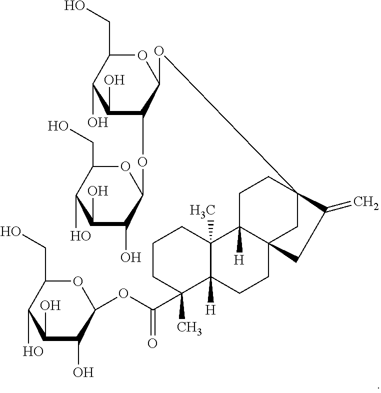

As used herein, the term “rebaudioside A” (CAS 58543-16-1) refers to (4α)-13-[(2-O-β-D-glucopyranosyl-3-O-β-Dglucopyranosyl-β-D-glucopyranosyl)-oxy]kaur-6-en-8-oic acid β-D-glucopyranosyl ester having the structure:

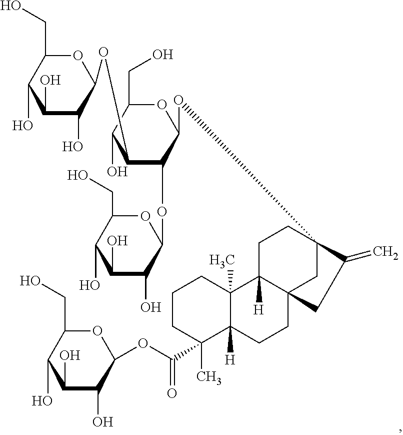

As used herein, the term “rebaudioside D” (CAS 63279-13-0) refers to (4α)-13-[(O-β-D-glucopyranosyl-(1→2)-O-[β-D-glucopyranosyl-(1→3)]-β-D-glucopyranosyl)oxy]kaur-16-en-18-oic acid 2-O-β-D-glucopyranosyl-β-D-glucopyranosyl ester having the structure:

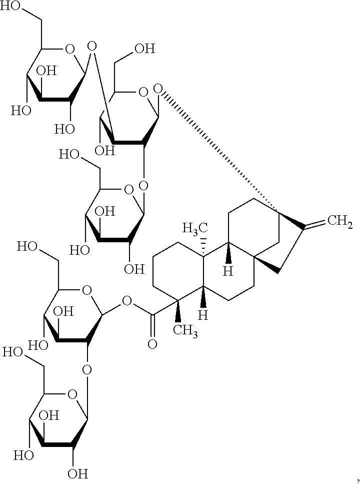

As used herein, the term “L-methylfolate” includes the compound (2S)-2-[[4-[(2-amino-5-methyl-4-oxo-1,6,7,8-tetrahydropteridin-6-yl)methylamino]benzoyl]amino]pentanedioic acid, having the CAS number 134-35-0, and pharmaceutically acceptable salts thereof.

As used herein, the term “N-acetylcysteine” includes the amino acid having the structure:

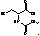

and pharmaceutically acceptable salts thereof.

### II. EMBODIMENTS

The present invention provides formulations and methods that increase the bioavailability of vitamin B₁₂ and other water-soluble compounds. The invention is based on the discovery that terpene glycosides can enhance the absorption of water-soluble compounds following oral administration. This enhancement can counteract the insufficient absorption caused by low levels of intrinsic factor and/or other plasma transporters as well as receptor defects in many vitamin B₁₂-deficient patients. The formulations and methods can be used to administer a variety of other water-soluble compounds for use as nutritional supplements, nutraceuticals, and/or therapeutic agents.

### III. FORMULATIONS FOR ENHANCING BIOAVAILABILITY

The present invention provides a formulation for enhancing bioavailability. The formulation includes a water-soluble vitamin and a glycoside selected from a diterpene glycoside or a triterpene glycoside.

In one embodiment, the present invention provides a formulation for enhancing the bioavailability of vitamin B₁₂, the formulation comprising:
vitamin B₁₂; and a glycoside selected from the group of a diterpene glycoside or a triterpene glycoside.

In certain aspects, the vitamin B₁₂ is a cobalamin selected from the group of cyanocobalamin, hydroxocobalamin, methylcobalamin, and 5-deoxyadenosylcobalamin. Preferably, the vitamin B₁₂ is methylcobalamin.

A. Active Ingredients

As described above, water-soluble vitamins include vitamin C (i.e., ascorbic acid) and the B vitamins. The B vitamins, in turn, include thiamine (vitamin B₁), riboflavin, niacin/nicotinic acid/nicotinamide (vitamin B₃), folic acid, folinic acid, L-methylfolate, L-5-methylfolate (vitamin B₉), pyridoxine/pyridoxal/pyridoxamine (vitamin B₆), biotin (vitamin B₇), pantothenic acid (vitamin B₅), and vitamin B₁₂.

The formulations of the present invention can also be used to enhance the bioavailability of a number of other water-soluble compounds. Examples of other water-soluble compounds include, but are not limited to, adenine, adenosine monophosphate, orotic acid, dimethylglycine, carnitine, choline, isoleucine, lysine, leucine, methionine, phenylalanine, threonine, tryptophan, valine, histidine, and pharmaceutically acceptable salts thereof.

Examples of water-soluble pharmaceuticals that can be included in the formulations of the invention include, but are not limited to, abacavir, acarbose, acetylcarnitine, acyclovir, albuterol, alendronic acid, alfacalcidol, alibendol, alprazolam, ambroxol, amiloride, amitriptyline, amlodipine, amphetamine, anastrozole, atenolol, atropine, azelastine, azulene, benazepril, benserazide, benznidazole, beraprost, biperiden, bisoprolol, brotizolam, bupropion, buspirone, cabergoline, camostat, capecitabine, captopril, cefaclor, cefcapene pivoxil, cefmetazole, cefroxadine, cetirizine, chloramphenicol, chlorpheniramine, choline alfoscerate, cilazapril, cimetidine, ciprofloxacin, citalopram, clomiphene, clomipramine, clonidine, cloxacillin, codeine, colchicine, cyclophosphamide, desloratadine, didanosine, diethylcarbamazine, digoxin, diltiazem, dolasetron, domperidone, donepezil, doxazosin, doxifluridine, enalapril, eperisone, epinastine, ergocalciferol, ergonovine, ergotamine, ethambutol, ethinyl estradiol, ethosuximide, etizolam, famciclovir, ferrous sulfate, fexofenadine, finasteride, fluconazole, fluoxetine, fluvastatin, fluvoxamine, fursultiamine, gabapentin, granisetron, hydralazine, imidapril, indapamide, isoniazid, isosorbide dinitrate, ketotifen, lamivudine, letrozole, levamisole, levetiracetam, levodopa, levofloxacin, levonorgestrel, levothyroxine, limaprost, lisinopril, lithium carbonate, lorazepam, losartan, loxoprofen, lumefantrine, mecobalamin, mefloquine, metformin, methotrexate, methyldopa, metoprolol, mexiletine, mirtazapine, montelukast, morphine, neostigmine, nicardipine, nicorandil, nifurtimox, nitroglycerin, nizatidine, norethindrone, norgestimate, olopatadine, ondansetron, oseltamivir, oxybutynin, pamidronate, paroxetine, penicillamine, pergolide, perindopril, phendimetrazine, phenoxymethylpenicillin, pilsicainide, pravastatin sodium, prednisolone, primaquine, procaterol, proguanil, promethazine, propiverine, propranolol, pseudoephedrine, pyrazinamide, pyridostigmine bromide, quetiapine, quinapril, rabeprazole, ramipril, ranitidine, reserpine, ribavirin, risedronic acid, rivastigmine, rizatriptan, rosiglitazone, saquinavir, sarpogrelate hydrochloride, sertraline, sildenafil, S-methylmethionine, stavudine, sumatriptan, tacrolimus, taltirelin, tamsulosin, tegafur, temocapril, tenofovir, disoproxil, terazosin, terbinafine, thioctic acid, tolterodine, topiramate, toremifene, tramadol, trimebutin, trimetazidine, tulobuterol, valacyclovir, venlafaxine, voglibose, zidovudine, zolmitriptan, zolpidem, and pharmaceutically acceptable salts thereof.

Some aspects of the invention provide a formulation for enhancing bioavailability, wherein the water-soluble vitamin is a B vitamin. In some aspects, the B vitamin is selected from B₁, B₃, B₅, B₆, B₇, B₉, B₁₂, and a combination thereof. In some embodiments, the B vitamin is vitamin B₁₂. In some aspects, the formulation is substantially free of intrinsic factor. In some aspects, the water-soluble vitamin is vitamin C.

In certain instances, the present invention provides a formulation or carrier comprising folate or folic acid, or a folic acid salt such as a pharmaceutically acceptable salt. In accordance with the present invention, the formulation comprises folic acid (pteroylmonoglutamate) or one or more folylpolyglutamates or compounds in which the pyrazine ring of the pterin moiety of folic acid or of the folylpolyglutamates is reduced to give dihydrofolates or tetrahydrofolates. Other suitable derivatives include the preceding compounds in which the N-5 or N-10 positions carry one carbon units at various levels of oxidation. Thus, examples of compounds suitable for use herein include, but are not limited to: folic acid (pteroylmonoglutamate), dihydrofolate, tetrahydrofolate, 5-methyltetrahydrofolate, 5,10-methylenetetrahydrofolate, 5,10-methenyltetrahydrofolate, 5,10-formiminotetrahydrofolate, 5-formyltetrahydrofolate (leucovorin), and 10-formyltetrahydrofolate. A preferred compound is folic acid. Each of the foregoing compounds and any pharmaceutically acceptable salt is also included.

B. Terpene Glycosides

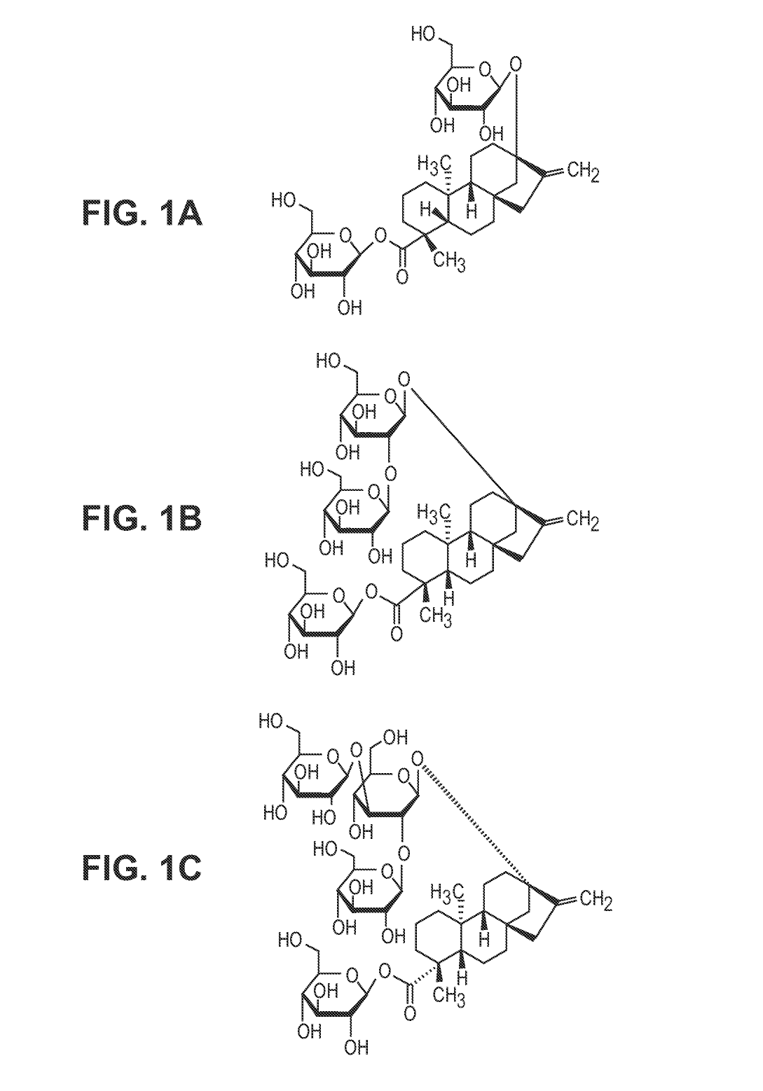

Natural terpene glycosides exist in a variety of plant sources. They generally are terpene aglycons attached to at least one glucose or other simple sugars (e.g., xylose or galactose), and the most common forms are monoterpene glycosides, diterpene glucosides, and triterpene glucosides. Many of these compounds arc known to be non-toxic and natural sweeteners. Examples of diterpene glycosides include rubusoside, rebaudioside, stevioside, and steviol monoside. Rubusoside A is a diterpene glycoside mainly from Chinese sweet leaf tea leaves (Rubus suavissimus; Rosaceae). Rubusoside A has a molecular formula C₃₂H₅₀O₁₃ and molecular weight of 642.73. The structure of rubusoside is shown in FIG. 1A. Rubusoside also has good solubility in water, alcohol and acetone ethyl acetate. The compound as shown in FIG. 1A is a diterpene aglycone with two glucose molecules attached.

Stevioside is a diterpene glycoside that is isolated from the Stevia leaf (Stevia rebaudiana; Asteraceae). Stevioside has a molecular formula C₃₈H₆₀O₁₈ and a molecular weight of 804. The structure is shown in FIG. 1B. The compound as shown is a diterpene aglycone with three glucose molecules. In pure form, it is a crystal or white powder. Another diterpene glycoside that is isolated from the Stevia leaf is rebaudioside, which exists in several forms, including rebaudioside A, rebaudioside B, rebaudioside C, rebaudioside D, rebaudioside E, and rebaudioside F. The structure of rebaudioside A is shown in FIG. 1C. The compound as shown is a diterpene aglycone with four glucose molecules. In pure form, it is a white powder.

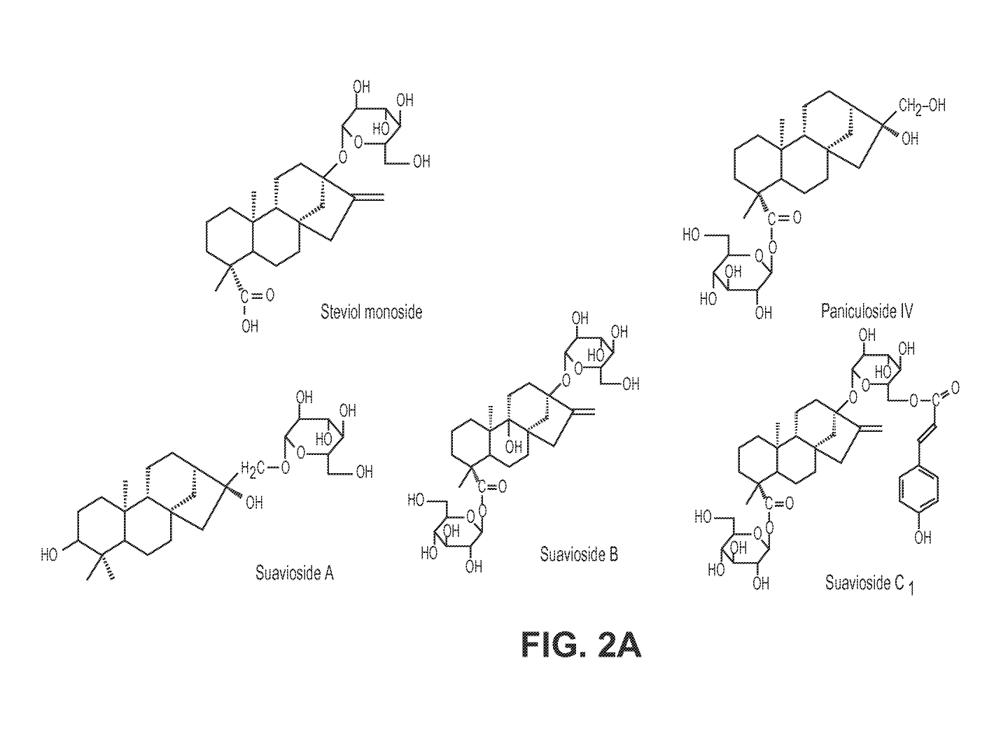

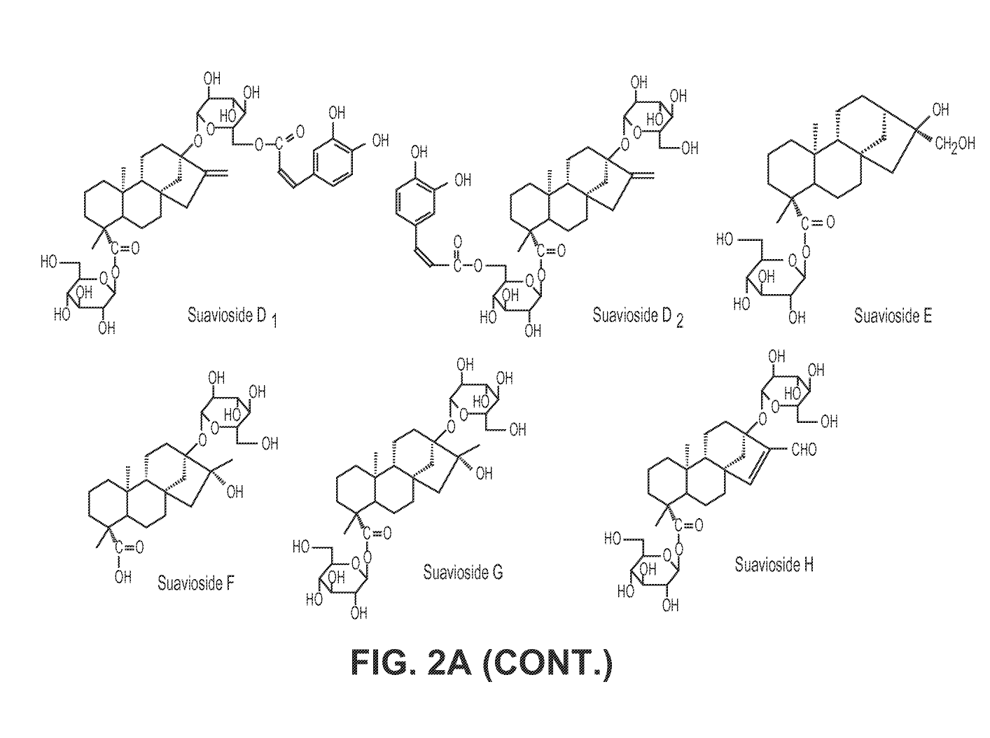

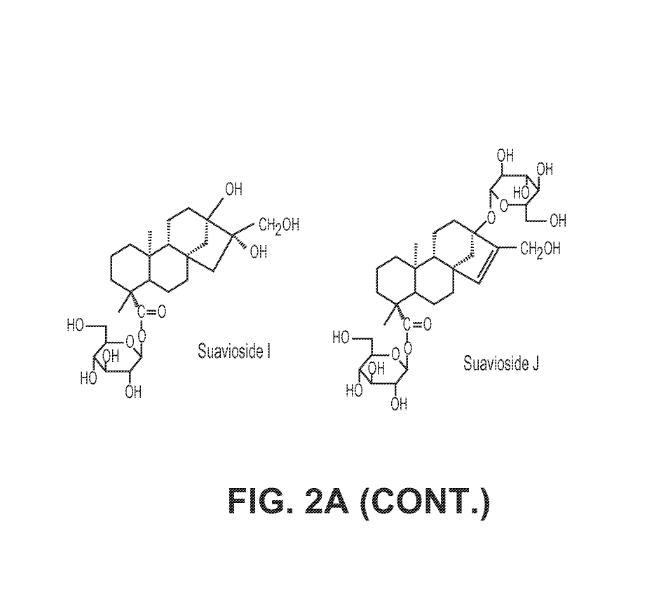

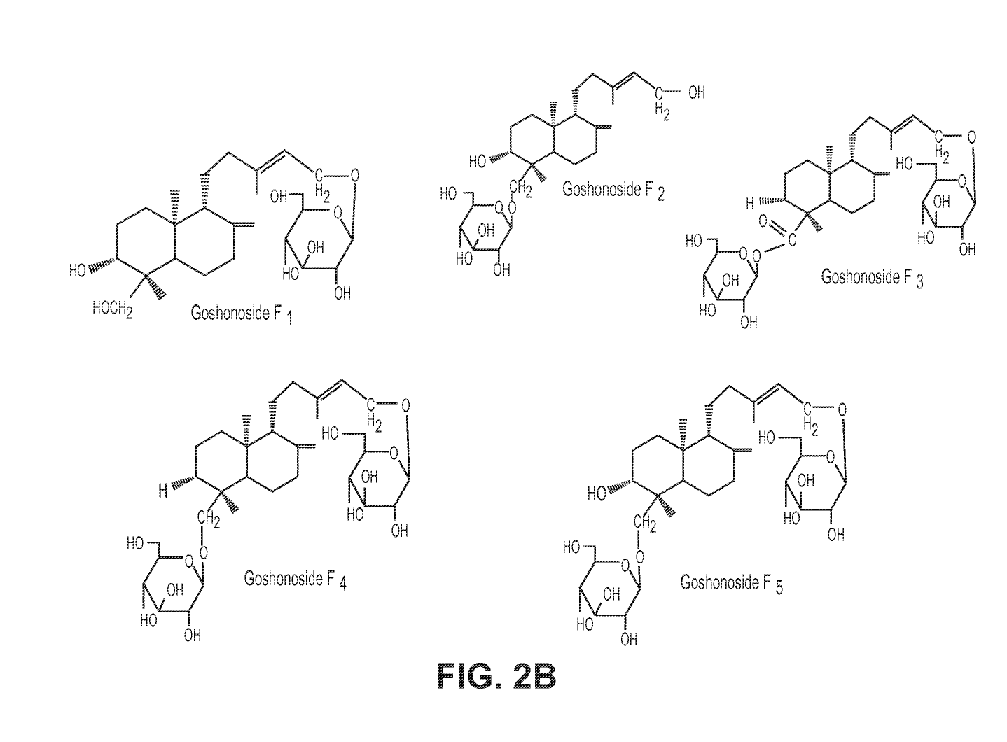

Another diterpene glycoside that is isolated from the Chinese sweet leaf tea (Rubus suavissimus; Rosaceae) and from stevia leaves (Stevia rebaudiana; Asteraceae) is steviol monoside. The structure of steviol monoside has only one glucose molecule (FIG. 2A) rather than two as in rubusoside. Steviol monoside can be isolated from the sweet leaf tea, stevia leaves, or be obtained through the partial acid or alkaline hydrolysis of rubusoside to cleave one glucose molecule. Unlike rubusoside, steviol monoside is not a dominant diterpene glycoside in the sweet leaf tea or stevia plant.

Other diterpene that contain various numbers of glucose moieties are known. These compounds include: paniculoside IV, suaviosides A, B, C₁, D₁, D₂, E, F, G, H, I, and J (FIG. 2A) as identified by Ohtani et al, (1992, Phytochemistry 31(5): 1553-1559), and goshonosides F₁ to F₅ (FIG. 2B) as identified by Seto et al. (1984, Phytochemistry 23 (12): 2829-2834). Although many diterpene glycosides such as stevioside, rebaudioside A, rubusoside, steviol monoside, and suavioside B, G, I, J, and H taste sweet, other diterpene glycosides are tasteless or bitter. For example, paniculoside IV is tasteless, suavioside C₁ tastes bitter, suavioside D₁ is tasteless, suavioside D₂ tastes bitter, suavioside E is tasteless, and suavioside F tastes bitter as indicated by Ohtani et at (1992, Phytochemistry 31(5): 1553-1559).

According some embodiments of the present invention provide a formulation for enhancing bioavailability as described above, wherein the glycoside is a diterpene glycoside. In some embodiments, the diterpene glycoside is selected from rubusoside, stevioside, rebaudioside A, rebaudioside B, rebaudioside C, rebaudioside D, rebaudioside E, rebaudioside F, steviol monoside, dulcoside A, steviol bioside, paniculoside, suavioside A, suavioside B, suavioside C1, suavioside D1, suavioside D2, suavioside E, suavioside F, suavioside G, suavioside H, suavioside I, suavioside J, goshonoside F1, goshonoside F2, goshonoside F3, goshonoside F4, and goshonoside F5. In some embodiments, the diterpene glycoside is selected from stevioside, rebaudioside A, and rebaudioside D. In some embodiments, the diterpene glycoside is stevioside.

Any suitable water-soluble vitamin or other water-soluble compound can be combined with any suitable terpene glycoside to prepare a formulation of the invention. For example, vitamin C or a B vitamin can be combined with a diterpene glycoside or a triterpene glycoside. A B-vitamin can be combined with a rebaudioside, a suavioside, goshonoside, rubusoside, or stevioside. Vitamin B₁₂ can be combined with a rebaudioside, suavioside, a goshonoside, rubusoside, or stevioside. Vitamin C can be combined with a rebaudioside, a suavioside, a goshonoside, rubusoside, or stevioside. In some embodiments, the water-soluble vitamin is vitamin B₁₂ and the diterpene glycoside is stevioside. Other combinations of water-soluble compounds and terpene glycosides can be used in the formulations of the invention.

Any suitable amount of terpene glycoside can be used in formulations of the present invention. In general, the amount of terpene glycoside is sufficient to increase the bioavailability of the water-soluble vitamin or other water-soluble substance. The ratio of the water-soluble vitamin to the terpene glycoside is typically from about 10:1 to about 1:500 by weight. The ratio of the water-soluble vitamin to the terpene glycoside can be for example, about 10:1, about 5:1, about 1:1, about 1:10, about 1:25, about 1:30, about 1:50, about 1:75. about 1:100, about 1:125, about 1:150, about 1:200, about 1:250, or about 1:500 by weight. In some embodiments, the ratio of the water-soluble vitamin to the terpene glycoside is from about 1:1 to about 1:100 by weight. In some embodiments, the ratio of the water-soluble vitamin to the terpene glycoside is from about 1:20 to about 1:50 by weight. In some embodiments, the ratio of the water-soluble vitamin to the terpene glycoside is from about 1:25 by weight. In some embodiments, the ratio of the water-soluble vitamin to the terpene glycoside is from about 1:25 to about 1:30 by weight.

In certain aspects, the formulation has a ratio of vitamin B₁₂: to the glycoside of 1:1 w/w to 1:100 w/w. In other aspects, the formulation has a ratio of vitamin B₁₂: to the glycoside of 1:20 w/w to 1:50 w/w. In other aspects, the formulation has a ratio of vitamin B₁₂: to the glycoside of 1:25 w/w to 1:30 w/w. In certain aspects, the formulation has a ratio of vitamin B₁₂: to the glycoside of 1:25 w/w.

In certain instances, the formulation is a complex or a particle such as a nanoparticle. In certain aspects, the present invention provides vitamin B₁₂ and a glycoside, which forms a nanoparticle that is about 0.1 nm to about 10 nm in diameter. In other aspects, the vitamin B₁₂ and glycoside form a nanoparticle that is about 1 nm to about 5 nm in diameter, such as 1 nm, 2 nm, 3 nm, 4 nm, or 5 nm.

In certain instances, the complex or particle of vitamin and glycoside is held together by Van der Waals forces, with no covalent bond formation.

In certain instances, the formulation is a polydisperse population of nanoparticles ranging from 1 nm to about 10 nm in diameter or 1 nm to about 5 nm in diameter.

In certain instances, the stevioside: methylcobalamin (MeCbl) complex comprises a nanoparticle, or a nanomicelle, with a characteristic size distribution of approximately 2 nm to about 4 nanometers. This size, along with their physical and chemical properties, is believed to be important regarding their bioavailability enhancement properties. Nanoparticie size distribution can be measured using Dynamic Light Scattering (DLS).

The formulations of the present invention can contain water-soluble vitamins in combination with each other, as well as with other vitamins, nutrients, and drug compounds. Some embodiments of the invention provide a formulation including vitamin B₁₂ as described above, further including L-methylfolate and vitamin B₆. In some embodiments, the formulation includes vitamin B₁₂, and further includes L-methylfolate and N-acetylcysteine. In some embodiments, the formulation includes vitamin B₁₂, and further includes L-methylfolate.

C. Formulations

Depending on the intended mode of administration, the pharmaceutical compositions can be in the form of a solid, a semi-solid or liquid dosage form, preferably in unit dosage form suitable for administration of precise dosages. In addition to the water-soluble vitamin and the terpene glycoside, the compositions can contain pharmaceutically-acceptable excipients. “Pharmaceutically acceptable excipient” refers to an excipient or mixture of excipients which does not interfere with the biological activity and bioavailability of the vitamins(s) and which is not toxic or otherwise undesirable to the subject to which it is administered. Since acceptable carriers and excipients are determined in part by the particular composition being administered as well as by the particular method used to administer the composition, there are a wide variety of suitable formulations of pharmaceutical compositions of the present invention (See, e.g., Remington's Pharmaceutical Sciences, 17^{th} ed., 1989).

For solid compositions, conventional excipients include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by dissolving, dispersing, etc., an active compound as described herein and optional pharmaceutical adjuvants in water or an aqueous excipient, such as, for example, water, saline, aqueous dextrose, and the like, to form a solution or suspension. If desired, the pharmaceutical composition to be administered may also contain minor amounts of nontoxic auxiliary excipients such as wetting or emulsifying agents, pH buffering agents and the like, such as sodium acetate, sorbitan monolaurate, triethanolamine acetate, and triethanolamine oleate. If desired, flavoring and/or coloring agents may be added as well. Other optional excipients for incorporation into an oral formulation include preservatives, suspending agents, thickening agents, and the like. Accordingly, some embodiments of the invention provide a formulation for enhancing bioavailability as described above, wherein the formulation is in the form selected from the group consisting of a capsule, a tablet, a softgel, a powder, an effervescent form, or a lozenge.

Injectable formulations can be prepared in conventional forms, either as liquid solutions or suspensions, solid forms suitable for solubilization or suspension in liquid prior to injection, or as emulsions or liposomal formulations. The sterile injectable formulation may also be a sterile injectable solution or a suspension in a nontoxic parenterally acceptable diluent or solvent. Among the acceptable vehicles and solvents that may be employed are water, Ringer's solution and isotonic sodium chloride solution. Solutions can contain antioxidants, buffers, bacteriostats, and solutes that render the formulation isotonic with the blood of the intended recipient. Aqueous and non-aqueous sterile suspensions can include suspending agents, solubilizers, thickening agents, stabilizers, and preservatives. Sterile, fixed oils, fatty esters or polyols can be employed as non-aqueous solvents or suspending media. Injection solutions and suspensions can also be prepared from sterile powders, granules, and tablets.

Suitable formulations for rectal administration include, for example, suppositories, which includes an effective amount of a packaged composition with a suppository base. Suitable suppository bases include natural or synthetic triglycerides or paraffin hydrocarbons. In addition, it is also possible to use gelatin rectal capsules which contain a combination of the composition of choice with a base, including, for example, liquid triglycerides, polyethylene glycols, and paraffin hydrocarbons.

### IV. METHODS FOR MAKING

In another embodiment, the present invention provides a method for making a formulation of vitamin B₁₂, the method comprising:
admixing vitamin B₁₂ and a glycoside selected from the group of a diterpene glycoside or triterpene glycoside in a solvent to form a vitamin B₁₂ admixture;
heating the vitamin B₁₂ admixture to form a clear vitamin B₁₂ admixture; and
removing the solvent from the clear vitamin B₁₂ admixture to form the formulation of vitamin B₁₂.

In certain aspects, the vitamin B₁₂ is a cobalamin selected from the group of cyanocobalamin, hydroxocobalamin, methylcobalamin, and 5-deoxyadenosylcobalamin. Preferably, the vitamin B12 is methylcobalamin.

In certain aspects, the formulation is substantially free of intrinsic factor.

In certain other aspects, the glycoside is a diterpene glycoside.

In certain aspects, the diterpene glycoside is a member selected from the group of rubusoside, stevioside, rebaudioside A, rebaudioside B, rebaudioside C, rebaudioside D, rebaudioside E, rebaudioside F, steviol monoside, dulcoside A, steviol bioside, paniculoside, suavioside A, suavioside B, suavioside C1, suavioside D1, suavioside D2, suavioside E, suavioside F, suavioside G, suavioside H, suavioside I, suavioside J, goshonoside F1, goshonoside F2, goshonoside F3, goshonoside F4, and goshonoside F5.

In certain aspects, the diterpene glycoside is a member selected from the group of stevioside, rebaudioside A and rebaudioside D. Preferably, the diterpene glycoside is stevioside.

In certain aspects, the formulation has a ratio of vitamin B₁₂: to the glycoside of 1:1 w/w to 1:100 w/w. In other aspects, the formulation has a ratio of vitamin B₁₂: to the glycoside of 1:20 w/w to 1:50 w/w. In certain other aspects, the formulation has a ratio of vitamin B1₂: to the glycoside of 1:25 w/w to 1:30 w/w or 1:20 w/w to 1:30 w/w such as 1:20, 1:21, 1:22, 1:23, 1:24, 1:25, 1:26, 1:27, 1:28, 1:29, or 1:30 w/w.

In certain aspects, the solvent is a C₁-C₆ alkanol such as methanol, ethanol, propanol, butanol, pentanol, hexanol, or a combination thereof. Other solvents include, but are not limited to, water, a C₃-C₈ ether, a C₃-C₆ ketone, a C₃-C₆ ester, or mixtures thereof.

In certain aspects, the reaction is carried out in the dark or with minimal light. Typically, the active ingredient or vitamin is added to a reaction flask and a solvent (an alkanol e.g. ethanol) is added. The ratio of active ingredient to solvent (e.g. alkanol) is about 1:1 to about 1:500, or 1:100 to about 1:500, or about 1:150 to about 1:300 or 1:250. Thereafter, the glycoside is added. The reaction mixture is heated to about 40° C. to about 100° C., or about 50° C. to about 80° C. or about 60° C. to about 80° C. After heating from about 1 minute to about 60 minutes the vitamin B₁₂ and glycoside admixture forms a clear vitamin B₁₂ admixture. Heating can be 1 minute, 5, 10, 15, 20, or 30 minutes. Removing the solvent (e.g., alkanol) forms a dry powder.

In certain aspects, the powder of vitamin B₁₂ and the glycoside is a nanoparticle. The nanoparticle has a size of about 0.1 nm to about 10 nm in diameter, or about 1 nm to about 5 nm in diameter.

A. Spray Drying

In general, spray drying methods employed herein produce a dry powder from a liquid suspension or slurry by rapidly drying with a hot gas or air. Typically, air or an inert as is the heated drying medium, and an atomizer or spray nozzle is used to disperse the liquid suspension or slurry into a controlled drop size spray. Typically, the spray drying drop sizes ranges from about 1 nm to 500 μm. In certain instances, the complexes produced herein are in the 1 nm to 10 μm diameter range. The dry powder that is generated is free-flowing.

With respect to Vitamin B₁₂, the solvent used for the slurry is typically water or an C₁-C₆ alkanol. In certain instances, the preferred solvent for spray drying Vitamin B₁₂ is ethanol. With respect to Vitamin B₉, the preferred solvent for spray-drying is water.

B. Batch Processing

In other instances, batch processing can be used to produce larger scale amounts of formulations of the present invention. Conventional batch processing is a cost-effective method for scaling-up manufacturing of small molecule active pharmaceutical ingredients. Reaction vessels are widely available in large sizes suitable for high throughput manufacturing. Solid products can generally be obtained by precipitation; resulting slurries are filtered to collect the desired product, typically using a filter pot or a centrifuge. Solid products, still wet with solvent, can be further dried using a variety of methods, the most common of which is tray-drying. With large reactors available for high throughput processes, economy of scale is generally realized and production costs can be minimized.

In this method, after the clear vitamin: stevioside complex is made, the solvent is removed typically by distillation. The distillation is carried out until the batch volume is lowered to approximately 20-25% of the original volume. Upon cooling, a precipitate is formed, resulting in stirred slurry.

The solvent such as ethanol, facilitates dissolution of the complex, which allows for complex formation. Typically, the complex a nanoparticle or nanomicelle. In certain instances, a solvent is used post-distillation, which facilitates precipitation and enhances the isolation of the desired product. The term “antisolvent” describes solvents in which a solid compound of interest has low solubility to facilitate precipitation. Antisolvents are commonly added to solutions to facilitate precipitation or crystallization of solid compounds. In certain instances, suitable antisolvents of the present invention include, but are not limited to, acetic acid, acetone, anisole, butanol, butyl acetate, tert-butylmethyl ether, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, propanol, propyl acetate, and mixtures thereof.

In certain instances, with approximately 20-30% of the original starting volume remaining, the complex slurry is treated with one or more antisolvents to enhance the precipitation process. The solid product can be tray-dried with heat and vacuum in a vacuum at 25 to 40° C. (e.g., 35° C.) for 24-48 hours. The product thus obtained is tested fix relative purity (e.g., reverse phase HPLC) and for residual solvent content e.g., gas chromatography).

The batch methods employing antisolvents are successful with regard to enhancing precipitation and providing solid product in high yield. Samples containing high levels of residual solvent tend to exhibit broad particle size distributions outside of the target range of 2-5 nm. Samples having low residual solvent tend to exhibit target particle size distribution. The residual solvent content in the complex is typically less than 10,000 ppm, or less than 8000 ppm, or even less than or equal to 5000 ppm.

C. In Situ Drying and Distillation

In other aspects, the present invention provides in situ distillation and drying methods, wherein the process is conducted essentially in a single vessel. For example, a horizontal plow mixer/dryer (HPMD) allows for mixing of solid materials during the drying stage.

In one aspect, a stevioside: methylcobalamin complex (stev: MeCbl) is prepared in a horizontal plow mixer/dryer (HPMD) at the kilogram scale. For example, the ratio of stevioside to MeCbl is 25:1 (wt/wt); the blending process involves dissolving the solid materials in an alcohol solvent such as ethanol, followed by evaporative removal of the solvent and drying of the resulting solid complex. The target level for residual solvent is less than 5000 ppm (0.5% wt/wt). In certain instances, the volume of ethanol used is 10 mL of ethanol per gram of stevioside (10 mL/g) or less, such as for example, 8 mL/g.

In one example, a stock solution of MeCbl, stevioside, and absolute ethanol is used. The solution can be prepared in a 22 L vessel or larger and exposure to light is avoided as MeCbl is light-sensitive in solution. The solution is charged into the vessel and the system is set up for distillation with heat on the jacket, cooling on the condenser and vacuum on the complete system. After much of the solvent is distilled, the volume of the solvent in the vessel decreases. The resulting solid product is further dried with heat and vacuum and in-process samples taken periodically to measure loss on drying (LOD). The methods produce free-flowing powder.

### V. METHODS FOR INCREASING THE BIOAVAILABILITY OF A WATER-SOLUBLE VITAMIN

In another embodiment, the present invention provides a method for increasing the bioavailability of a water-soluble vitamin. The method includes administering a formulation containing a water-soluble vitamin and a glycoside selected from a diterpene glycoside and a triterpene glycoside, thereby increasing the bioavailability of the water-soluble vitamin.

The methods of the invention can include administering any of the formulations described above so as to increase the bioavailability of the water-soluble vitamin. The increase in the bioavailability can be observed by any suitable means, including as an increase in the area under the curve (AUC) for a plot of the vitamin's circulation concentration over time after administration of the formulation. Accordingly, some embodiments of the invention provide a method for increasing the bioavailability of a water-solubility vitamin, wherein the water-soluble vitamin has increased bioavailability (AUC) as a result of the formulation. As described above, the methods of the present invention generally lead to bioavailability increases of at least about 10%, as compared to administration via control methods. The bioavailability can be increased, for example, by 25%, or 50%, or 100% (i.e., by 1 fold). The bioavailability can be increased by 200% (i.e., by 2 fold). In some embodiments, the bioavailability of the water-soluble vitamin is increased between about 1 to 10 fold over a control. In some aspects, the bioavailability of the water-soluble vitamin is increased between about 2 to 6 fold over a control. The control methods typically include administration of vitamin formulations that do not contain terpene glycosides.

In certain instances, the bioavailability of vitamin B₁₂ is increased between about 1 to 10 such as 1, 2, 3, 4, 5, 6, 7, 8, 9, or 10 fold over a control. In other instances, the bioavailability of vitamin B₁₂ is increased between about 2 to 6 fold over a control.

Frequency of administration of the formulations described herein, as well as dosage, will vary from individual to individual, and may be readily established using standard techniques. A suitable dose is an amount of a formulation that, when administered as described above, is capable of providing a beneficial level of a water-soluble vitamin or other water-soluble compound in the circulation of the subject to whom the formulation is administered. Those of skill in the art are aware of the routine experimentation that will produce an appropriate dosage range for a subject in need of treatment. Those of skill are also aware that results provided by in vitro or in vivo experimental models can be used to extrapolate approximate dosages for a patient in need of treatment.

### VI. EXAMPLES

### Example 1

### Preparation of Vitamin B₁₂ Formulations

Vitamin B₁₂ (methylcobalamin) is a dark reddish powder. Stevioside Extract 90% ST (“stevia”) is a white powder. Formulations were prepared under minimum light at room temperature.

One gram of vitamin B₁₂ powder was weighed and placed in an empty 2-liter evaporating flask. 250 mL of absolute ethanol was added to the flask. The vitamin B₁₂ to ethanol ratio was about 1:250 w/v. The mixture was shaken slightly at room temperature until the majority of vitamin B₁₂ dissolved.

25 grams of stevia powder was weighed and added directly to the vitamin B₁₂ ethanol solution. The weight ratio of stevia to vitamin B₁₂ was 25:1 w/w. The stevia concentration in ethanol was 10% w/v.

The flask containing both vitamin B₁₂ and stevia in ethanol was immediately heated in a water bath at 60-80° C. until the stevia was completely dissolved (around 3 minutes). The ethanol vitamin B₁₂-colored solution containing vitamin B₁₂ and stevia appeared clear and transparent.

As soon as the clarity of the ethanol solution was achieved, the ethanol solution was evaporated under a rotary evaporator (Buchi Rotavapor Collegiate Model) to completely remove the ethanol. Rotary evaporation was conducted using a water bath at 60° C., a vacuum pressure between 200 and 300 mbar, and an appropriate rotation speed up to a maximum of 280 rotations per minute. When all ethanol was removed, the vitamin B₁₂/stevia mixture became dry and solid.

A free-flowing powder was obtained by scooping out the dried and solid material with a scalpel or similar tool. Grinding to desired particle size is possible and optional. The resulted vitamin B₁₂-stevia powder had a vitamin B₁₂ content of 3.85% by weight.

An initial solubility test of the vitamin B₁₂-stevia powder was conducted. 52 mg of the vitamin B₁₂ powder was added to 250 mL of water and shaken slightly for a minute or two. A transparent solution formed with the vitamin B₁₂ color (slight reddish at this concentration).

The powder was sealed in a glass bottle and stored at room temperature with good ventilation and in darkness. Cold storage (4° C.) was also attempted without any observed benefit based on the physical properties of the product.

The formulations in Table 1 were prepared according to the general procedure described above.

TABLE 1
Vitamin B₁₂ Formulations

| Formulation Name | Vitamin B₁₂:Stevia Ratio (w:w) |
|---|---|
| F1 | 1:25 |
| F2 | 1:50 |
| F3 | 1:125 |
| F4 | 1:0 (no stevia; control) |

The vitamin B₁₂ was successfully complexed with stevia at weight to weight ratios of 1:25, 1:50, and 1:125 between the vitamin B₁₂ and stevia. The vitamin B₁₂-stevia complex was made to powder form.

### Example 2

### Stability of Vitamin B₁₂ in Gastric Fluid

The pH of simulated gastric fluid was measured. Then F1 and F4 were prepared in this fluid to a concentration of 20 μg/mL. The solutions were incubated at 37° C. in the water bath of a shaker operated at 50 rpm. After 2 hrs. of incubation, 1 mL solution was taken for HPLC analysis.

The vitamin B₁₂-stevia complex powder was instantly and completely reconstituted in water or simulated gastric fluid to concentrations of up to 4 mg/mL vitamin B₁₂ in the presence of 100 mg/mL stevia. The reconstituted water or gastric fluid solution was completely dilutable by a factor of 40, 100, or 200.

In gastric fluid, the formulation F1 (at 1:25 w/w ratio) was reconstituted. The pure vitamin B₁₂ (F4) was also reconstituted in gastric fluid as an unformulated control. Both sample solutions were made to contain 20 μg/ml vitamin B₁₂, wrapped with aluminum foil to avoid exposure to light, and incubated at 37° C. for two hours. The solutions were then stored in a freezer for 22 hrs. The frozen samples were thawed to room temp before HPLC-UV analysis. The vitamin B₁₂ was stable in gastric fluid over a two hour period with or without stevia (Table 2).

TABLE 2
Stability of vitamin B₁₂ in simulated gastric fluid for 2 hours.

| Determined | | | |
|---|---|---|---|
| Concentration (μg/mL) | | R.S.D. (%) | R.E. (%) |
| | 2 hr | 2 hr | 2 hr |

| 0 hr | F1 | F4 | F1 | F4 | F1 | F4 |
|---|---|---|---|---|---|---|
| 20.2 | 19.5 | 21.0 | 2.1 | 7.0 | −3.6 | 3.7 |

R.S.D.—relative standard deviation;
R.E.—relative error.

### Example 3

### Absorption Assessment in Caco-2 Cell Monolayer

A Caco-2 cellular model was used to study the effect of the stevia solubilizer on the intestinal absorption of vitamin B₁₂. The Caco-2 cell monolayer model is the pharmaceutical industry standard for predicting human oral absorption. The test samples included:

Formula 1 (F1): vitamin B₁₂ in 0.5 mg/mL stevia; Formula 2 (F2): vitamin B₁₂ in 1 mg/mL stevia; Formula 3 (F3): vitamin B₁₂ in 2.5 mg/mL stevia; and Formula 4 (F4): vitamin B₁₂ alone (Control).

Using the Caco-2 cell monolayer assay, it was found that the vitamin B₁₂ was not permeable by itself. The formulations using stevia did not have promoting effects on its permeability across the cell monolayer (Table 3), which mimics the intestinal absorption. It is suspected that the Caco-2 cellular model may not be suitable for detecting absorption enhancement since it mimics the intestinal conditions, by passing the actions that might be taking place under the conditions of the stomach, which might be important for vitamin B 12 in particular.

TABLE 3
In Vitro ADME-Tox Summary

| | | | | Assay | Mean A-> | |
|---|---|---|---|---|---|---|
| | Sample | | Analyte | Duration | B Pₐₚₚ^{a} | |
| Sample | Concentration | | Test Conc. | (hr) | (10⁻⁶ cm s⁻¹⁾ | Comment |
| Ranitidine | 10 | μM | 10 μM | 2 | 0.3 | Low permeability, |
| | | | | | | efflux |
| Warfarin | 10 | μM | 10 μM | 2 | 40.1 | High permeability |
| | | | | | | control |
| Ranitidine | 10 | μM | 10 μM | 2 | 0.3 | Low permeability, |
| | | | | | | efflux |
| Warfarin | 10 | μM | 10 μM | 2 | 39.7 | High permeability |
| | | | | | | control |
| F1 | 0.02 | mg/mL | 20 μg/mL | 2 | <LLOQ^{c} | |
| F2 | 0.5 | mg/mL | 20 μg/mL | 2 | <LLOQ^{c} | |
| F3 | 1 | mg/mL | 20 μg/mL | 2 | <LLOQ^{c} | |
| F4 | 2.5 | mg/mL | 20 μg/mL | 2 | <LLOQ^{c} | |

^{a}Pₐₚₚ—apparent permeability.
^{b}Assay replicate
^{c}LLOQ—lower limit of quantification; compound not detected in receiver side.

### Example 4

### Absorption Assessment in a Rat Model

To answer the question whether the solubilizer enhanced the absorption of vitamin B₁₂, the four formulations were assessed in a Sprague Dawley rat model at the vitamin B₁₂ dose equivalent to 1 mg/70 kg person. Each group (formulation) had 6 rats (n=6). Each rat was orally gavaged and blood collected at 11 time points during a 12-hour period. The plasma samples were stored in a freezer prior to vitamin B₁₂ analyses. Relative bioavailability was calculated by comparing the area under the time-concentration curves of formulated over the unformulated control.

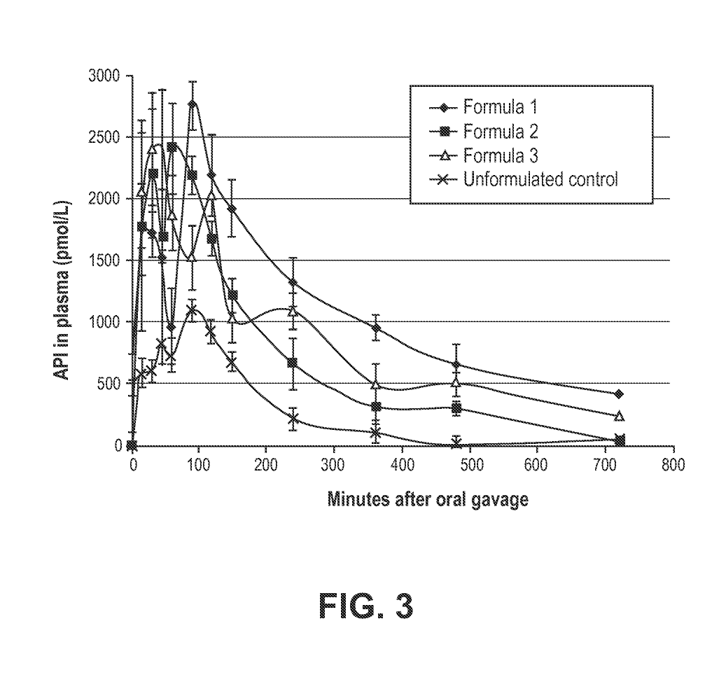

Vitamin B₁₂ in rat plasma over time after oral gavage with baseline normalized for all treatments were analyzed by a radioimmunoassay method. The vitamin B₁₂ in rat plasma over a 12-hr period at 15, 30, 45, 60, 90 min, and 2, 2.5, 4, 6, 8, and 12 h after oral gavage at a dose equivalent to 2 mg/70 kg person in a free form (unformulated control) or a formulated form of Formula 1 (vitamin B₁₂:stevia at 1:25 w/w), Formula 2 (vitamin B₁₂:stevia at 1:50 w/w), or Formula 3 (vitamin B₁₂:stevia at 1:125 w/w) were analyzed. Compared to the unformulated control, AUC was increased by 3.2 fold, 1.6 fold, or 2.2 fold, through the use of the absorption enhancer at 1:25, 1:50, or 1:125 ratios, respectively (FIG. 3). Cₘₐₓ (maximum plasma concentration) was significantly increased when the absorption enhancer was used. Tₘₐₓ (the time at which Cₘₐₓ is reached) remained the same at 90 min between the unformulated control and Formula 1. Formulas 2 and 3 with higher ratios of enhancer (1:50 and 1:125) tended to shorten the Tₘₐₓ in a dose-response fashion to 60 min (50-ratio) and 45 min (125-ratio). Vitamin B₁₂ in plasma was detected during the first 480 min (8 hrs.) for the unformulated control whereas it lasted for 720 min (12 hrs.) for the formulated vitamin B₁₂, especially Formula 1 (1:25). Extended absorption was shown and could last even longer beyond the experimental period of 12 hrs. If all sources (diet plus supplement) of vitamin B₁₂ are considered, vitamin B₁₂ absorption was increased by 40%, 20%, 28% corresponding to the use of the absorption enhancer at 25-ratio (Formula 1), 50-ratio (Formula 2), or 125-ratio (Formula 3), respectively. If the source of diet was normalized as “baseline,” vitamin B₁₂ absorption was increased to 4.2 fold, 2.6 fold, or 3.2 fold for the absorption enhancer at 25-ratio, 50-ratio, or 125-ratio, respectively. As demonstrated, stevia was effective for vitamin B₁₂ absorption. By using the absorption enhancer at the 50 mg dose, the absorption of vitamin B₁₂ via the current vitamin B₁₂ product can be increased by over 4 fold.

### Example 5

### Spray-Drying B₁₂ Stevioside

The sprayed-dried powder was produced by a lab-scale spray dryer, which produced a small average particle size with minimal agglomeration. During spray drying, the solution was maintained at 70° C. for a few hours. The product is easily spray-dried and produces good yield.

The following procedure for making the B₁₂ spray-dry solution was as follows: a ratio of 1:25 methylcobalamine: stevioside was used. 79.2 g of H₂O in an aluminum covered beaker was used. The water was heated and maintain at 70° C. Thereafter 21.51 g stevioside was added and allowed to dissolve (15-30 min). Thereafter, 0.8 grams of methylcobalamine was added and stirred (15 min). The solution stayed stable (no phase separation, no precipitation) for more than 24 hours.

The water content (by Karl-fisher method) of the spray-dried powder in an aqueous solvent is about 4.40% water. If ethanol is used, the water content is lower.

### Example 6

### Solvent Exchange Batch Procedure

This example shows a 25:1 Stevia:Methylcobalamin complex formation. The experimental set-up used was a 2 L, single-neck, flask equipped with a magnetic stirrer and a rotary evaporator or rotovap. To the flask wrapped completely in foil for protection from light was charged 25.0 g of stevia, 10 g of methylcobalamin and 260 mL of ethanol, 200 Proof. The mixture was agitated at ambient temperature for 15 minutes. The flask was placed on a rotovap and ˜125 mL of solvent removed at <35° C. under reduced pressure. Thereafter, five separate solvent exchanges were performed each with 250 mL of Solvent X (antisolvent) and continued to remove the solvent at <35° C. Next, the thick slurry was reduced to a volume of ˜250 mL. The thick slurry at ambient temperature was agitated for one hour. The solids were filtered through a medium, sintered-glass funnel. Next, rinse the solids with 100 mL of Solvent X. Place the solids in a vacuum oven at 35° C. for at least 48 hours. Yields were essentially quantitative.

| | | | Solvent |
|---|---|---|---|
| Expt Number | Solvent X | Ethanol Content | X Content |
| 01 | Heptane (200 g run) | 3700 ppm | 62200 ppm |
| 01A | Acetone | 6757 ppm | 36651 ppm |
| 01B | EtOAc | 297 ppm | 29699 ppm |
| 01C | IPAc | 180 ppm | 34801 ppm |
| 101A | MTBE | 21430 ppm | 45715 ppm |
| 101B | IPA | 122 ppm | 50261 ppm |

The experiments with antisolvent proved successful in regard to enhancing precipitation and providing solid product in high yield. Particle size distribution was and found to meet the desired particle size specification. Example DLS data from experiments using heptane antisolvent are found in Table 4.

The experiments and testing revealed a correlation between residual solvent levels and particle size distribution. Samples containing high levels of residual solvent tend to exhibit broad particle size distributions outside of the target range (2-5 nm). Samples low in residual solvent tend to exhibit target particle size distribution. These experiments also validated the batch process as a method which can produce good quality Stev:MeCbl complex without the formation of unwanted by-products.

TABLE 4
Particle size distribution analysis of Stev:MeCbl from heptane
precipitation.

| Particle | | | | |
|---|---|---|---|---|
| size | | | | |
| based | Sample | | | |
| on mean | conc | | | |
| number | (mg/mL) | 225-B | 4-01 | 8-01 |
| | ST 50 | 244.0 | 3.1 | 2.9 |
| | ST 50 | 275.0 | 2.4 | 2.5 |
| Avg | ST 50 | 259.5 | 2.8 | 2.7 |
| particle | | | | |
| size (nm) | | | | |
| | ST 100 | 343.0 | 2.5 | 3.1 |
| | ST 100 | 411.0 | 2.5 | 2.7 |
| Avg | ST 100 | 377.0 | 2.5 | 2.9 |
| particle | | | | |
| size (nm) | | | | |
| Note | | Large particles | Most stable | Stable for |
| | | formed within | after | ~3 hrs after |
| | | 5 min of | reconstinition | reconstitution |
| | | reconstitution | (longer than | |
| | | | 5 hrs) | |

### Example 7

### In Situ Drying and Distillation

This example illustrates in situ drying and distillation.

This example involves the preparation of 2×1 Kg batches of stev:MeCbl complex. The stock solution was 0.095 Kg of MeCbl, 2.125 Kg of stevioside, and 14.35 Kg of absolute ethanol. The solution was prepared in a 22 L vessel and exposure to light was avoided as MeCbl is highly light-sensitive in solution. The stock solution was stored in two containers and protected from light. The first run utilized half of the stock solution and the second run used the remaining portion.

The total stock solution weight was approximately 16 Kg. Approximately 8 Kg of stock solution was used for run 1. The solution for run 1 was charged in portions due to the limited capacity of the vessel. Initially, 2.05 Kg of solution was charged and the system was set up for distillation with heat on the jacket, cooling on the condenser and vacuum on the complete system. After much of the solvent distilled, volume in the vessel decreased. The vacuum was released and more stock solution was charged to the vessel (low light conditions). Distillation resumed and the cycle repeated until half (approximately 8 Kg) of the original stock solution was processed. The resulting solid product was further dried with heat and vacuum and in-process (IP) samples were taken periodically to measure loss on drying (LOD). Run 1 was stopped after LOD reached 2.4%. Free-flowing powder product was removed and weighed (360 g). Some residual product remained in the vessel, stuck to the plows and vessel wall. This material was kept in the vessel with the aim of recovering after completion of run 2.

Run commenced with a 2 Kg charge of the second half (approximately 8 Kg of original 16 Kg) of the stock solution. The cycle was repeated until the remaining stock solution was processed. Run 2 was stopped when the in-process sampling revealed 0.7% LOD. The vessel was discharged of free-flowing powder (660 g) and then product stuck in the vessel was removed, providing an additional 630 g of solid product.

Run 1 distillation time was approximately 4 hours and drying time was approximately hours. Run 2 distillation time was approximately 6 hours and drying time was approximately 4 hours. The highest temperature on the jacket during drying, for both runs, was approximately 166-169° F.

Run 1 powder, run 2 powder and brushout (scrapings) were kept separate. HPLC analyses of the batches indicated high quality products with little or no generation of new impurities. GC analyses revealed residual ethanol. These results demonstrated that process successfully produced high quality product.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

1. A formulation for enhancing bioavailability of vitamin B₁₂, the formulation comprising:
vitamin B₁₂; and
a glycoside selected from the group consisting of a diterpene glycoside and a triterpene glycoside.

2. The formulation of claim 1, wherein the vitamin B₁₂ is a cobalamin selected from the group consisting of cyanocobalamin, hydroxocobalamin, methylcobalamin, and 5-deoxyadenosylcobalamin.

3. The formulation of claim 2, wherein the vitamin B₁₂ is methylcobalamin.

4. The formulation of claim 1 wherein the formulation is substantially free of intrinsic factor.

5. The formulation of claim 1, wherein the glycoside is a diterpene glycoside.

6. The formulation of claim 5, wherein the diterpene glycoside is selected from the group consisting of rubusoside, stevioside, rebaudioside A, rebaudioside B, rebaudioside C, rebaudioside D, rebaudioside E, rebaudioside F, steviol monoside, dulcoside A, steviol bioside, paniculoside, suavioside A, suavioside B, suavioside C1, suavioside D1, suavioside D2, suavioside E, suavioside F, suavioside G, suavioside H, suavioside I, suavioside J, goshonoside F1, goshonoside F2, goshonoside F3, goshonoside F4, and goshonoside F5.

7. The formulation of claim 6, wherein the diterpene glycoside is selected from the group consisting of stevioside, rebaudioside A, and rebaudioside D.

8. The formulation of claim 7, wherein the diterpene glycoside is stevioside.

9. The formulation of claim 1, wherein the formulation has a ratio of vitamin B₁₂ to the glycoside of 1:1 w/w to 1:100 w/w.

10. (canceled)

11. The formulation of claim 1, wherein the formulation has a ratio of vitamin B₁₂ to the glycoside of 1:25 w/w to 1:30 w/w.

12. The formulation of claim 1, wherein said vitamin B₁₂ and said glycoside form a nanoparticle that is about 1 nm to about 10 nm in diameter.

13. (canceled)

14. (canceled)

15. A method for making a formulation of vitamin B₁₂, said method comprising:
admixing vitamin B₁₂ and a glycoside selected from the group consisting of a diterpene glycoside and a triterpene glycoside in a solvent to form a vitamin B₁₂ admixture;
heating the vitamin B₁₂ admixture to form a clear vitamin B₁₂ admixture; and
removing the solvent from the clear vitamin B₁₂ admixture to form the formulation of vitamin B₁₂.

16. The method of claim 15, wherein the vitamin B₁₂ is a cobalamin selected from the group consisting of cyanocobalamin, hydroxocobalamin, methylcobalamin, and 5-deoxyadenosylcobalamin.

17. The method of claim 16, wherein the vitamin B₁₂ is methylcobalamin.

18. The method of claim 15, wherein the formulation is substantially free of intrinsic factor.

19. The method of claim 15, wherein the glycoside is a diterpene glycoside.

20. The method of claim 19, wherein the diterpene glycoside is selected from the group consisting of rubusoside, stevioside, rebaudioside A, rebaudioside B, rebaudioside C, rebaudioside D, rebaudioside E, rebaudioside F, steviol monoside, dulcoside A, steviol bioside, paniculoside, suavioside A, suavioside B, suavioside C1, suavioside D1, suavioside D2, suavioside E, suavioside F, suavioside G, suavioside H, suavioside I, suavioside J, goshonoside F1, goshonoside F2, goshonoside F3, goshonoside F4, and goshonoside F5.

21. The method of claim 20, wherein the diterpene glycoside is selected from the group consisting of stevioside, rebaudioside A, and rebaudioside D.

22. The method of claim 21 wherein the diterpene glycoside is stevioside.

23. The method of claim 15, wherein the formulation has a ratio of vitamin B₁₂ to the glycoside of 1:1 w/w to 1:100 w/w.

24. (canceled)

25. The method of claim 15, wherein the formulation has a ratio of vitamin B₁₂ to the glycoside of 1:25 w/w to 1:30 w/w.

26. The method of claim 15, wherein the solvent is a C₁-C₆ alkanol or water.

27. The method of claim 15, wherein the solvent is removed from the clear vitamin B₁₂ solution by spray-drying or by in situ distillation.

28. (canceled)

29. (canceled)

30. The method of claim 15, wherein vitamin B₁₂ and said glycoside form a nanoparticle that is about 1 nm to about 10 nm in diameter.

31. (canceled)

32. A method for increasing the bioavailability of vitamin B₁₂, the method comprising:
orally administering to a human a formulation comprising vitamin B₁₂; and a glycoside selected from the group consisting of a diterpene glycoside and a triterpene glycoside; thereby increasing the bioavailability of vitamin B₁₂ to the human.

33. The method of claim 32, wherein the glycoside is a diterpene glycoside.

34. The method of claim 33, wherein the diterpene glycoside is selected from the group consisting of rubusoside, stevioside, rebaudioside A, rebaudioside B, rebaudioside C, rebaudioside D, rebaudioside E, rebaudioside F, steviol monoside, dulcoside A, steviol bioside, paniculoside, suavioside A, suavioside B, suavioside C1, suavioside D1, suavioside D2, suavioside E, suavioside F, suavioside G, suavioside H, suavioside I, suavioside J, goshonoside F1, goshonoside F2, goshonoside F3, goshonoside F4, and goshonoside F5.

35. The method of claim 34, wherein the diterpene glycoside is selected from the group consisting of stevioside, rebaudioside A, and rebaudioside D.

36. The method of claim 35, wherein the diterpene glycoside is stevioside.

37. (canceled)

38. The method of claim 32, wherein the formulation has a ratio of vitamin B₁₂ to the glycoside of 1:1 w/w to 1:100 w/w.

39. (canceled)

40. The method of claim 32, wherein the formulation has a ratio of vitamin B₁₂ to the glycoside of 1:25 w/w to 1:30 w/w.

41. (canceled)

42. The method of claim 32, wherein the bioavailability of vitamin B₁₂ is increased between about 1 to 10 fold as compared to a control containing the same amount of vitamin B₁₂, but lacking the glycoside.

43. The method of claim 42, wherein the bioavailability of vitamin B₁₂ is increased between about 2 to 6 fold as compared to the control.
